(19) United States
(12) Patent Application Publication
(10) Pub. No.: US 2005/0245998 A1
(43) Pub. Date: Nov. 3, 2005

(54) Hand held pulse laser for therapeutic use

(75) Inventors: Ralph Pruitt, Parker, CO (US); Gerry Graham, Centennial, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: LED Healing Light, LLC, Aurora, CO (US)

(21) Appl. No.: 11/118,296

(22) Filed: Apr. 29, 2005

Related U.S. Application Data
(60) Provisional application No. 60/566,881, filed on Apr. 30, 2004.

(51) Int. Cl. A61N 5/067

(52) U.S. Cl. 607/89

(57) ABSTRACT

A pulse laser for therapeutic use including a housing sized to be hand held by an operator. All components of the pulse laser are located within or on the housing. Thus, the present invention is a completely hand held stand alone unit which may be operated without a tethered connection to any apparatus located outside of the housing. The components located within the housing or on the housing include a laser light source, a control circuit configured to cause the laser light source to emit pulsed laser light, and a power supply. The wavelength of light produced by the laser light source may be about 635 nm. The control circuit of the therapeutic pulse laser may provide for multiple user selectable pulse rates. The therapeutic pulse laser may include a semiconductor switch in electrical communication with the control circuit and the laser light source. Ideally, the semiconductor switch will provide for active sourcing of current to the laser light source and active draining of current from the laser light source. The therapeutic pulse laser may also include an apparatus allowing for the exchange of digital information between the pulse laser and an external apparatus such as a database, computer, or second pulse laser unit.

### RELATED APPLICATION DATA

This application claims benefit of commonly assigned U.S. Provisional Patent Application Ser. No. 60/566,881, entitled HAND HELD PULSE LASER FOR THERAPEUTIC USE, filed Apr. 30, 2004, which application is incorporated herein by reference in its entirety.

### TECHNICAL FIELD

The present invention is directed toward a pulse laser for therapeutic use, and more particularly toward a hand held untethered pulse laser.

### BACKGROUND ART

Light has a profound effect on the human body. Light therapies have proved beneficial in the areas of pain management, and can further be used to specifically target individual pathogens or treat tissue dysfunctions or wounds. Light applied in a therapeutic manner can be either from a full or broad spectrum source or from a controlled source, such as a laser, which provides monochromatic light over a relatively narrow range of wavelengths.

Light emitting diodes (LEDs) have been used to provide a therapeutic monochromatic light source. LED light therapy units for consumer or home use have been developed in recent years. LED units are particularly suitable for consumer devices since LEDs have low power requirements and therapeutic LED light sources can be made which are simple and easy for consumers to operate and use. The primary drawback to LED based therapeutic light sources is that LEDs produce light which, although monochromatic, is diffuse in its projection. Laser diodes, on the other hand, can produce a coherent beam of light which may be focused or collimated and directed specifically to targeted areas.

Much research on the use of laser light of various frequencies has been directed toward the use of specific wavelengths to kill pathogens as a substitute for the use of antibiotics. In addition, laser light can be utilized to stimulate the body's own defense mechanism to kill pathogens and enhance other body physiology. Specific wavelengths of light may increase cellular reproduction, increase micro and macro cellular drainage functions, clear functional imbalances of the central nervous system, and even change cellular structure.

Thus, laser light in select wavelengths applied to the human body for therapeutic use can be used to treat conditions such as RSD, closed head injury, fibromyalgia, endocrine dysfunction such as PMS, low back pain, neck pain, and other conditions. Significant benefit has been observed when the light applied in therapy is pulsed at a select frequency.

Laser diodes, as opposed to LEDs, have rather substantial power requirements. In addition, the output of laser diodes, if not carefully controlled, can be harmful. Accordingly, commercially available pulse lasers for therapeutic use typically have a hand held laser unit connected by a flexible cord to a separate control/power supply unit. Commercially available therapeutic pulse lasers are thus typically bulky, expensive, and somewhat difficult to use.

Prior art therapeutic pulse lasers typically rely on a simple connection to ground to drain current from an active laser diode. Passive current draining from a laser diode takes time. The amount of time necessary for a laser diode to transition from a fully illuminated state to a fully off state depends upon the nature of the laser diode and the associated circuitry. However, the decay time associated with the passive draining of current from an activated laser diode is often the factor which limits the maximum pulse rate. High pulse rates are desirable for certain therapeutic treatments. It is often impossible to achieve a suitably high laser pulse rate using passively drained laser driver circuitry. Prior art devices relying on passive current draining technologies may be limited to pulse rates of 300 kHz or less.

In addition, passive current drain from a laser diode will allow the light output from the laser to decay over a period of time which is characteristic of the laser diode and associated circuitry. Thus, the passive draining of current from a laser diode makes it difficult to achieve a pulse with a sharply defined end point. As discussed above, a pulse with a sharply defined end point, which can be graphically represented as a square wave, may have significant therapeutic influences on the human body.

Certain therapeutic pulsed laser based treatment regimens have been found to provide beneficial treatment to human patients. The treatment regimens can be somewhat complex. A great deal of operator time may be necessary to program and reprogram complex treatment regimens. In addition, the possibility of programming error is increased when treatment regimens are manually programmed to a therapeutic pulse laser. Prior art therapeutic lasers typically do not have the functional capability to rapidly upload or download therapeutic regimens or other data to or from a centrally accessible database.

The present invention is directed toward overcoming one or more of the problems discussed above.

### SUMMARY OF THE INVENTION

One aspect of the present invention is a pulse laser for therapeutic use including a housing sized to be hand held by an operator. All components of the pulse laser are located within or on the housing. Thus, this aspect of the present invention is a completely hand held and stand alone unit which may be operated without a tethered connection to any apparatus located outside of the housing. The components located within the housing or on the housing include a laser light source, a control circuit configured to cause the laser light source to emit pulsed laser light, and a power supply.

An input keypad with buttons or switches to provide specific control functions may be operatively associated with the therapeutic pulse laser and located on the housing. The input keypad will be in electrical communication with the control circuit. Similarly, a display may be located on the housing to show the operator various operational parameters and assist with the programming and control of the therapeutic pulse laser. The display will also be in electrical communication with the control circuit.

The wavelength of light produced by the laser light source may be about 635 nm. This wavelength has been shown to provide specific therapeutic benefits when applied to the human body. The power supply, which is located within the hand held housing, will typically be a rechargeable battery.

The control circuit of the therapeutic pulse laser may provide for multiple user selectable pulse rates. The multiple user selectable pulse rates may be programmed directly by an operator through the input keypad, or previously downloaded or stored user selectable pulse rates may be activated or initiated by the operator through use of the keypad. The laser light source may include an array of multiple diode lasers. In this embodiment, at least two of the multiple diode lasers which make up the array may be pulsed at multiple and independent user selectable pulse rates.

The therapeutic pulse laser also includes a semiconductor switch in electrical communication with the control circuit and the laser light source. The semiconductor switch will provide for active sourcing of current to the laser light source and active draining of current from the laser light source. This configuration will allow for improved pulse frequency response since the decay time associated with a passive current drain from the laser light source is minimized. A suitable semiconductor switch will provide for a pulse frequency greater than 300 kHz. Pulse frequencies exceeding 1 MHz are possible. A representative semiconductor switch which provides for the active draining of current and the active sourcing of current is a power MOSFET half bridge.

The therapeutic pulse laser may also include an apparatus allowing for the exchange of digital information between the pulse laser and an external apparatus such as a database. Similarly, data could be exchanged between two separate pulse laser units. The data exchange apparatus also provides for the convenient programming of the therapeutic pulse laser. For example, various different therapeutic pulse regimens might be downloaded from a central database to an individual hand held unit through the apparatus for exchanging information. The apparatus for exchanging information may be of any type known in the computing arts, however, the use of a removable storage medium associated with the housing is particularly well suited for the implementation of this embodiment of the therapeutic pulse laser.

Another aspect of the present invention is a pulse laser for therapeutic use including a laser light source, a control circuit configured to cause the laser light source to emit pulsed laser light, and a semiconductor switch in electrical communication with the control circuit. The semiconductor switch provides for active sourcing of current to the laser light source and the active draining of current from the laser light source. A power MOSFET half bridge is one example of a semiconductor switch which is suitable for providing active sourcing and active draining of current to and from the laser light source. A suitable semiconductor switch in conjunction with the control circuit may provide for a pulse rate in excess of 1 MHz.

Another aspect of the present invention is a method of providing therapy including providing a therapeutic pulse laser which is sized to be hand held by an operator, and which includes a laser light source and a power supply. The therapeutic pulse laser is configured to be operated without a tethered connection to another apparatus. The method of providing therapy also includes applying pulsed laser light to a select portion of a patient's body to achieve a specific therapeutic purpose.

The method of providing therapy may also include controlling the pulse rate of the pulsed laser light by actively sourcing current to the laser light source and actively draining current from the laser light source, thus achieving a highly controlled, extremely rapid pulse rate with laser light pulses having well defined end points.

The laser light source may include multiple diode lasers. In such a case, the method may further include applying the pulsed laser light to select portions of a patient's body at multiple and independent operator selectable pulse rates. The pulsed laser light applied to the patient may have a wavelength of about 635 nm.

The method of providing therapy may also include exchanging information between the therapeutic pulse laser and a separate apparatus. The separate apparatus may be a computer, database, or a second therapeutic pulse laser unit. Information may be exchanged through removable storage medium, a wireless connection, a wired connection plugged into a suitable data port associated with the pulse laser, or by other means recognized in the data processing or computer arts.

### BRIEF DESCRIPTION OF THE DRAWINGS

FIG. 1 is a top plan view of a hand held pulse laser consistent with the present invention;

FIG. 2 is a perspective view of a hand held pulse laser consistent with the present invention showing the relative size of an embodiment of the invention;

FIG. 3 is a block diagram of an embodiment of a hand held pulse laser consistent with the present invention;

FIG. 4 is an exploded perspective view of a hand held pulse laser consistent with the present invention showing a laser light source including multiple diode lasers; and

FIG. 5 is a perspective view of a hand held pulse laser consistent with the present invention engaged with a charging stand.

### DETAILED DESCRIPTION OF THE PREFERRED EMBODIMENT

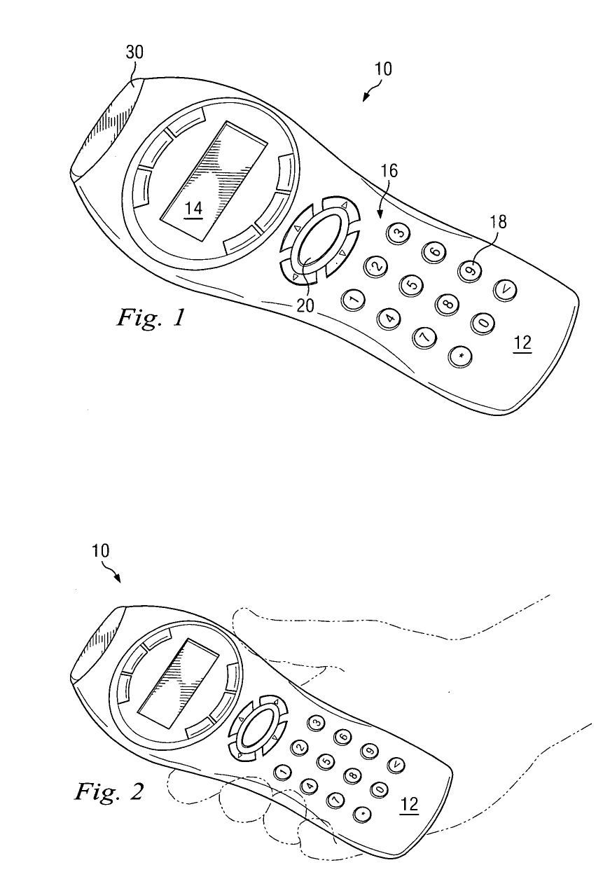

The pulse laser for therapeutic use, referred to herein as “pulse laser” 10 includes various components contained within or on a housing 12. As shown in FIG. 1 and FIG. 2, the housing 12 is sized to be comfortably held in the hand of an operator. A display panel 14 is associated with the exterior of the housing 12. The display 14 can be used to display the operational status of the pulse laser 10 and can, in conjunction with an input keypad 16, be used to control the operation of the pulse laser 10.

Individual keys or buttons of the input keypad 16 can be associated with specific operation and control tasks. Representative examples of individual buttons used to control the operation of the pulse laser 10 include an on/off switch; a timer switch, useful for setting the duration of a pulse lasing treatment; and a light switch, used to backlight the display 14 for ease of visibility.

In addition, certain other input buttons of the input keypad 16 are preferably not associated with specific operational functions but are available to specifically program or set certain user designed or user accessed therapeutic lasing protocols to be executed by the pulse laser 10. In particular, scroll buttons, a cancel button, a select button, and delete button can all be used to maneuver through and select user operational and control menus displayed on the display 14. These buttons used in conjunction with a numeric keypad 18 can be used by an operator of the pulse laser 10 to select, modify, and deselect specific therapeutic protocols or regimens. The selected therapeutic protocols can be user designed, pre-programmed, manufactured, or downloaded to the pulse laser 10. The input keypad 16 may also include a laser pulse button 20 which allows an operator to manually pulse therapeutic laser light or initiate a selected therapeutic protocol.

The specific nature or configuration of the input keypad 16 used to control and operate the pulse laser 10 can be varied. The overall configuration of the housing 12 is selected so that the entire pulse laser 10 is self contained and is easily hand held, and the input keypad 16 is easily manipulated by the operator. Specific contours can be molded or otherwise fabricated into the housing 12 to achieve an ergonomically appropriate shape for hand held use.

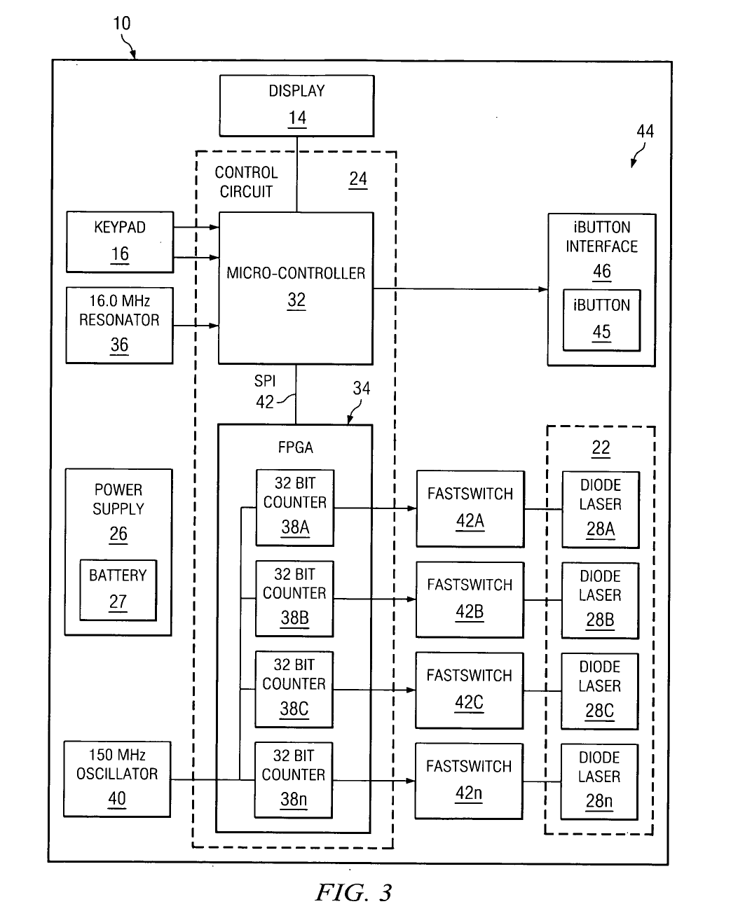

The therapeutic pulse laser 10 includes all components necessary for untethered operation within or on the housing 12. In particular, as shown in the block diagram of FIG. 3, a laser light source 22 and a control circuit 24 are operatively disposed on or within the housing 12. The control circuit 24 is in electronic communication with the laser light source 22 and configured to cause the laser light source 22 to emit pulsed laser light. Also included within the housing 12 is a power supply 26. Typically, the power supply 26 will be a rechargeable battery 27 such as a lithium ion battery, lithium polymer battery, or other type which is selected to provide a suitable voltage and amperage for operation of the control circuit 24, display 14, and laser light source 22, while being sized small enough to fit within the housing 12. In addition, it is desirable that any battery 27 associated with the power supply 26 be readily and easily recharged as described in detail below.

The operative elements of the laser light source 22, control circuit 24, and power supply 26 are illustrated in block diagram form in FIG. 3. The laser light source 22 may include an array of diode lasers 28. The array as shown in FIG. 3 includes four diode lasers 28A, 28B, 28C . . . 28n, however, any suitable number of individual diode lasers 28 may be selected to form an array.

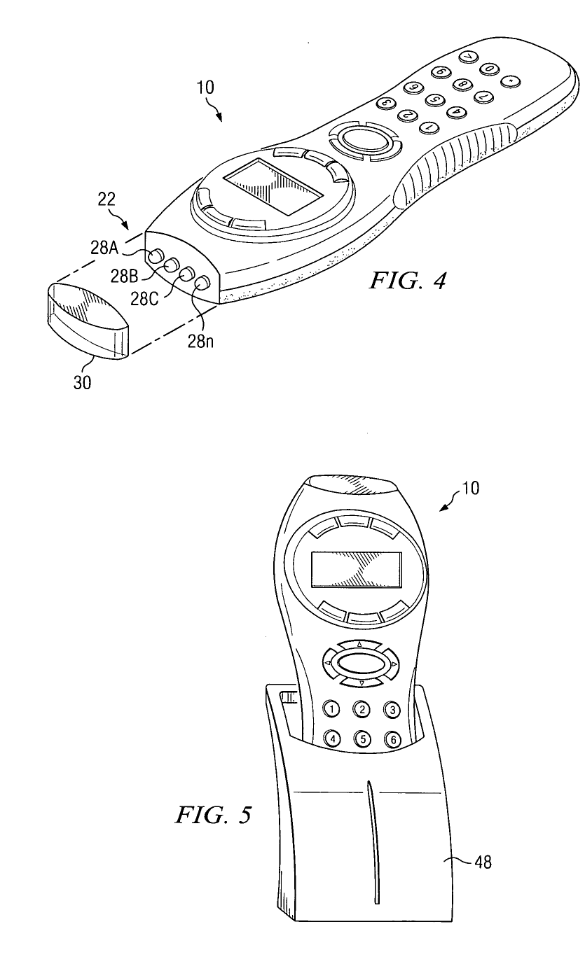

In the views of FIG. 1 and FIG. 2, the laser light source 22, in particular the array of individual diode lasers 28A, 28B, 28C . . . 28n, is not visible as the laser light source 22 is positioned behind a guard 30 attached to the housing 12. In the exploded exterior perspective view of FIG. 4, the guard 30 has been removed and the laser light source 22, in particular an array of four diode lasers 28A, 28B, 28C . . . 28n is visible.

The geometric arrangement or focal direction of the diode lasers 28 included in the laser light source 22 can be selected to achieve specific therapeutic goals. Thus, the output from individual diode lasers 28A, 28B, 28C . . . 28n may be applied at different angles or different locations with respect to a treatment subject to achieve therapeutic goals. In addition, it is desirable that the control circuit 24 provide for the user selection of a suitable pulse rate from multiple possible pulse rates. Ideally, the individual diode lasers 28 of the laser light source 22 may be pulsed at multiple and independent user selectable pulse rates.

Various types of laser diodes 28 are suitable for use with the pulse laser 10. A particularly suitable type of laser diode 28 is a 5 mw class 3A laser diode operating near the wavelength 635 nm. Optionally, the laser diodes 28 can be associated with selected lenses or filters to focus or modify the output light. The preferred wave length of 635 nm falls within the red range of visible light and both provides some heating therapy and other benefits. This wavelength is readily transmitted through the skin to deeper tissues. Other wavelengths can be employed to achieve specific therapeutic benefits. Preferably, any individual laser diode 28 in the laser light source 22 can be independently pulsed at a user selectable pulse rate ranging from 0.1 Hz to 150.0 MHz and higher frequencies, ideally with accuracy up to 0.1% for pulse frequencies under 10 KHz, and accuracy approaching 1% on frequencies under 100 KHz. The pulsed light can be delivered as a sine pulse or a digital square wave pulse to achieve specific therapeutic benefits. The generation of a suitable square wave pulse with a well defined end point and minimal decay time is discussed in detail below.

Also included within the housing 12 is a control circuit 24. In one embodiment of the therapeutic pulse laser 10, as depicted in FIG. 3, the control circuit 24 includes a microcontroller 32 in communication with a field programmable gate array 34. The microcontroller 32 receives input from a clock or resonator 36. Similarly, the field programmable gate array 34, which includes a series of counters 38A, 38B, 38C . . . 38n, receives input from a second clock or resonator 40. The oscillating input signal from the resonator 36 and oscillator 40 may be modified by timers associated with the microcontroller 32 or the counters 38A, 38B, 38C . . . 38n to generate a suitable pulsed output signal to drive the laser light source 22.

The control circuit 24, and specifically the microcontroller 32, also receives user input from the input keypad 16 and outputs information to the display 14. It should be noted that the components depicted in FIG. 3 and described herein are one example of a suitable control circuit 24. Although this configuration is suitable for control of the output and functions of a therapeutic pulse laser 10 as described herein, other suitable circuits may be devised. The present invention is not limited to the configuration depicted in FIG. 3.

Output from timers associated with the microcontroller 32, after amplification, could be sent directly to the laser light source 22 for the generation of pulsed laser light output. However, it has been determined that driving the laser light source 22 directly from the voltage and/or current amplified output of readily available microcontrollers 32 may limit the frequency response of the pulse laser 10. Accordingly, it is desirable to take the output from the microcontroller 32 and feed it into a separate field programmable gate array 34 which includes one or more 32 bit timers 38A, 38B, 38C . . . 38n. The timers 38 of the field programmable gate array 34 may receive input from a separate oscillator 40 which will allow for much faster timing frequencies, and ultimately increased output frequency response. The timers 38 of the field programmable gate array 34 may be loaded from the microcontroller 32 using an serial parallel interface (SPI) 42 or other suitable bus or connection.

The microcontroller 32 will preferably have programmable flash memory in addition to data processing circuitry. Many types of suitable onboard microcontrollers 32 are available commercially. For example, an ATmega32 microcontroller by ATMEL Corporation is a suitable microcontroller for the control of the pulse laser 10. The present invention is not limited to this controller, however. The present invention may be implemented with any suitable control circuit.

Typically, the one or more laser diodes 28 selected for the laser light source 22 will require more power than is required by the control circuit 24 or the microcontroller 32. In addition, it is desirable that the lasers be pulsed on and off at a high frequency with high accuracy. The power and switching requirements of the laser light source 22, and in particular laser diodes 28A, 28B, 28C . . . 28n, can be met by supplying each laser diode 28 power through a semiconductor switch 42. In one possible configuration of the invention, Analogitech AAT4900 MOSFET buffered power half bridge devices have been shown to be suitable for powering and switching the laser diodes 38. Other suitable semiconductor switch 42 packages are readily available.

High frequency pulsing in excess of the 300 kHz pulse rate of certain prior art devices and more accurate pulse width control can be achieved if the semiconductor switch 42 both actively sources current to a diode laser 28 and actively drains current from a diode laser 28. Active sourcing and draining of current to and from the diode laser 28 minimizes the passive output decay associated with passive draining methods such as merely grounding one leg of a given diode laser 28 and provides for pulse rates in excess of 1 MHz. The minimization of output decay associated with passive current draining thus allows for the generation of an output pulse having a well defined end point. Accordingly, the use of a semiconductor switch 42 which provides for the active sourcing and draining of current allows the production of an output pulse which has a substantially square wave form. A square wave output with a well defined end point is both potentially therapeutic and provides for significantly higher pulse frequencies before the individual nature of each pulse is lost.

Ideally, the voltage applied to the semiconductor switch 42 is regulated by a low dropout linear regulator such as a Texas Instruments TPS76601. Other voltage regulators would also be suitable for use in the output electronics of the pulse laser 10.

Various therapeutic regimens can be programmed to the microcontroller 32 by use of the input keypad 16. However, manual programming can be time consuming and may result in an error. It is preferable to download treatment regimens to the microcontroller 32 from a database associated with a separate apparatus. Accordingly, it is desirable to provide the pulse laser 10 with an apparatus for exchanging information 44 between the pulse laser and an external apparatus such as a computer, database, or another pulse laser 10. Various types of suitable apparatus for exchanging information 44 may be associated with the pulse laser 10 and contained within the housing 12 or located on the housing 12. For example, the apparatus for exchanging information 44 may be removable storage media such as a memory stick, a miniature diskette or tape, or as is shown in FIG. 3, the apparatus for exchanging information 44 may be an iButton 45 communicating with an iButton interface 46 in communication with the microcontroller 32. Alternatively, the apparatus for exchanging information 44 may be a wireless data transmitter operating with infrared, radio, or other wireless technology associated with the microcontroller 32. The apparatus for exchanging information could be as simple as a data port such as a USB, parallel, or serial port operatively associated with the housing 12 and communicating with the microcontroller 32. In such an implementation, the data port would be configured to receive a data cable for wired connection to an exterior computer, database, or second pulse laser 10.

The apparatus for exchanging information 44 will provide for information to be downloaded to the pulse laser 10, or for information to be uploaded from the pulse laser 10 to a central database. For example, complicated treatment regimens may be downloaded from a central database to the pulse laser 10. Similarly, treatment regimens developed by practitioners and found to be useful could be exchanged among practitioners over the internet. In addition, updates to the functional capabilities of the pulse laser 10 could be downloaded to the pulse laser 10 through the apparatus for exchanging information 44.

When the pulse laser 10 is in use, power is supplied to the control circuit 24 and diode lasers 28 by an onboard power supply 26. Preferably, the power supply 26 will include a battery 27, typically a lithium ion, lithium polymer, or other type of battery 27 which can be quickly and repeatedly recharged. Preferably, the battery 27 can be removed from the housing 12 of the pulse laser 10 and swapped with a fresh battery 27 so that no down time is experienced if recharging becomes necessary while the pulse laser 10 is in use.

As shown in FIG. 5, the battery 27 may be charged in an external charging stand 48 similar to those used for other hand held devices such as cellular phones, thus the battery 27 may be charged while attached to the pulse laser 10. Alternatively, a receptacle may be provided in the housing 12 for connection of a conventional charging unit jack to the pulse laser 10.

Proper charging and discharging of the battery 27 may be controlled by the combined actions of a battery management circuit located within the housing 12 and the control circuit 24. A battery management circuit can optimize the battery 27 functioning and can extend the battery 27 lifetime. In addition, power-down functions may be controlled by the control circuit 24. For example, the control circuit 24 may cause the pulse laser to become dormant after a period of inactivity. Preferably, the battery management circuit is implemented as an integrated circuit such as an Analogitech AAT3680 battery manager.

Battery 27 output will be used to drive both the laser light source 22 and the control circuit 24. Integrated control circuitry typically requires a highly regulated 5 volt DC power source. Output from the battery can be regulated for these purposes with a DC voltage regulator such as a Texas Instruments TPS76350 low power, low dropout voltage regulator.

Another aspect of the present invention is a method of providing therapy using a therapeutic pulse laser 10. Since the pulse laser 10 is sized to be hand held by an operator and includes an internal laser light source 22 and power supply 26, the pulse laser 10 may be operated without a tethered connection to another apparatus. The untethered nature of the pulse laser 10 affords an operator or user of the device a great deal of freedom in moving the pulse laser 10 over a patient's body and positioning the pulse laser with respect to a patient's body. As discussed in detail above, pulsed laser light may be applied to select portions of a patient's body according to preprogrammed regimens, or the output of the pulse laser 10 may be directly controlled through the input keypad 16.

Certain treatment regimens may be best implemented if laser light from multiple laser diodes 28 is pulsed at more than one pulse rate and simultaneously applied to select portions of the patient's body. This functionality can be achieved with the pulse laser 10 as described herein by selecting multiple and independent pulse rates for more than one of the multiple diode lasers 28A, 28B, 28C . . . 28n which are included in the laser light source 22. Similarly, a very high pulse rate, in excess of 300 kHz, may be achieved with the therapeutic pulse laser 10 as described herein by actively sourcing current to the laser light source 22 and actively draining current from the laser light source 22 by means of a suitably selected semiconductor switch 42A, 42B, 42C . . . 42n associated with each diode laser 28A, 28B, 28C . . . 28n.

The ease of preparing to use the pulse laser 10 to provide therapy may be enhanced by exchanging information between the therapeutic pulse laser 10 and a separate apparatus through the apparatus for exchanging information 44. In particular, various treatment protocols or regimens may be uploaded or downloaded to the pulse laser from a computer, database, or second pulse laser 10, thus eliminating the time, inconvenience, and potential for error associated with manual programming through the input keypad 16.

The objects of the invention have been fully realized through the embodiments disclosed herein. Those skilled in the art will appreciate that the various aspects of the invention may be achieved through different embodiments without departing from the essential function of the invention. The particular embodiments are illustrative and not meant to limit the scope of the invention as set forth in the following claims.

1. A pulse laser for therapeutic use comprising:
a housing sized to be hand held;
a laser light source operatively located within the housing;
a control circuit disposed within the housing in electronic communication with the laser light source and configured to cause the laser light source to emit pulsed laser light;
a power supply in electronic communication with the laser light source, the power supply being operatively located within the housing allowing the pulse laser to be operated without a tethered connection to any apparatus located outside of the housing and;
a semiconductor switch in electronic communication with the control circuit and the laser light source, wherein the semiconductor switch provides for active sourcing of current to the laser light source and active draining of current from the laser light source.

2. The pulse laser of claim 1 further comprising an input keypad on the housing, the input keypad being in electronic communication with the control circuit.

3. The pulse laser of claim 1 further comprising a display on the housing, the display being in electronic communication with the control circuit.

4. The pulse laser of claim 1 wherein the wavelength of light produced by the laser light source is about 635 nm.

5. The pulse laser of claim 1 wherein the power supply comprises a rechargeable battery.

6. The pulse laser of claim 1 wherein the control circuit provides for multiple user selectable laser pulse rates.

7. The pulse laser of claim 1 wherein the laser light source further comprises an array of multiple diode lasers.

8. The pulse laser of claim 7 wherein at least two of the multiple diode lasers may be pulsed at multiple and independent user selectable pulse rates.

9. The pulse laser of claim 1 wherein the semiconductor switch comprises a power MOSFET half-bridge.

10. The pulse laser of claim 1 wherein the semiconductor switch in conjunction with the control circuit provide for a pulse rate in excess of 300 kHz.

11. The pulse laser of claim 1 wherein the semiconductor switch in conjunction with the control circuit provide for a pulse rate in excess of 1 MHz.

12. The pulse laser of claim 1 further comprising means for exchanging information between the pulse laser and a separate apparatus, the means for exchanging information being in communication with the control circuit.

13. The pulse laser of claim 12 wherein the means for exchanging information comprises a removable storage medium.

14. A pulse laser for therapeutic use comprising:
a laser light source;
a control circuit in electronic communication with the laser light source and configured to cause the laser light source to emit pulsed laser light; and
a semiconductor switch in electronic communication with the control circuit and the laser light source providing for active sourcing of current to the laser light source and active draining of current from the laser light source.

15. The pulse laser of claim 14 wherein the semiconductor switch comprises a power MOSFET half-bridge.

16. The pulse laser of claim 14 wherein the semiconductor switch in conjunction with the control circuit provide for a pulse rate in excess of 300 kHz.

17. The pulse laser of claim 14 wherein the semiconductor switch in conjunction with the control circuit provide for a pulse rate in excess of 1 MHz.

18. A method of providing therapy comprising:
providing a therapeutic pulse laser which is sized to be hand held by an operator, which includes a laser light source, a control circuit and a power supply and which therapeutic pulse laser may be operated without a tethered connection to another apparatus, the therapeutic pulse laser further including a semiconductor switch in electronic communication with the control circuit and the laser light source providing for active sourcing of current to the laser light source and active draining of current from the laser light source; and
applying pulsed laser light to a select portion of a patient's body.

19. The method of providing therapy of claim 18 further comprising applying pulsed laser light to a select portion of a patient's body at a pulse rate exceeding 300 kHz.

20. The method of providing therapy of claim 18 further comprising applying pulsed laser light to a select portion of a patient's body at a pulse rate exceeding 1 MHz.

21. The method of providing therapy of claim 18 wherein the laser light source comprises multiple diode lasers, further comprising applying the pulsed laser light to the select portion of the patient's body at multiple and independent operator selectable pulse rates.

22. The method of providing therapy of claim 18 further comprising exchanging information between the therapeutic pulse laser and a separate apparatus.

23. The method of providing therapy of claim 18 wherein the pulsed laser light applied to the patient has a wavelength of about 635 nm.
